(19) United States
(12) Patent Application Publication
(10) Pub. No.: US 2014/0066743 A1
(43) Pub. Date: Mar. 6, 2014

(54) OBJECT INFORMATION ACQUIRING APPARATUS

(71) Applicant: CANON KABUSHIKI KAISHA, Tokyo (JP)

(72) Inventors: Takao Nakajima, Kyoto-shi (JP); Yasufumi Asao, Kyoto-shi (JP)

(73) Assignee: CANON KABUSHIKI KAISHA, Tokyo (JP)

(21) Appl. No.: 14/010,701

(22) Filed: Aug. 27, 2013

(30) Foreign Application Priority Data
Sep. 4, 2012 (JP) 2012-193844

(51) Int. Cl. A61B 5/00

(52) U.S. Cl. CPC A61B 5/0095
USPC 600/407

(57) ABSTRACT

An object information acquiring apparatus is used which includes a first light source outputting irradiation light delivered to an object, an output unit to which the irradiation light is guided and which includes an opening smaller than a wavelength of the irradiation light, a probe detecting an acoustic wave generated when the object absorbs near-field light output by the output unit, and a signal processing unit acquiring information on an interior of the object from the acoustic wave detected by the probe.

### BACKGROUND OF THE INVENTION

1. Field of the Invention

The present invention relates to an object information acquiring apparatus.

2. Description of the Related Art

In general, imaging apparatuses using X rays, ultrasound waves, or magnetic resonance imaging (MRI) are commonly used in the field of medicine. On the other hand, in the field of medicine, active studies have been conducted on optical imaging apparatuses which obtain in vivo information by allowing light output by a light source such as a laser to propagate through an object such as a living organism and detecting the propagated light or the like. As one of such optical imaging techniques, a photoacoustic imaging technique has been proposed.

In the photoacoustic imaging, first, an object is irradiated with pulsed light generated by a light source, and a living tissue having absorbed the energy of light propagating and diffusing in the object generates an acoustic wave (hereinafter referred to as a photoacoustic wave), which is detected at a plurality of positions. Then, the resultant signals are analyzed to visualize information related to an optical characteristic value for the interior of the object. Thus, the distribution of the optical characteristic value for the interior of the object, particularly the distribution of an optical energy absorption density, can be obtained.

Examples of a photoacoustic detector include a transducer using a piezoelectric phenomenon and a transducer using a change in capacity, and in recent years, a detector using optical resonance has been developed.

Furthermore, Japanese Patent Application Laid-open No. 2007-307007 (Patent Literature 1) uses near-field light as pulsed light to detect a photoacoustic signal generated by an optical absorber located in a segment close to a light source.

On the other hand, there has been a demand to increase imaging resolution in order to allow a smaller optical absorber to be imaged using a photoacoustic technique. Thus, development of a photoacoustic microscope configured to increase the resolution of photoacoustic imaging by focusing sound or pulsed light has been promoted. According to Japanese Translation of PCT Application No. 2011-519281 (Patent Literature 2), the resolution is improved by focusing pulsed light by a lens and placing an object at the focal position of the light. Japanese Translation of PCT Application No. 2011-519281 discloses that a resolution of the order of 1.0 micrometer (μm) can be achieved.

Patent Literature 1: Japanese Patent Application Laid-open No. 2007-307007

Patent Literature 2: Japanese Translation of PCT Application No. 2011-519281

### SUMMARY OF THE INVENTION

However, according to Japanese Translation of PCT Application No. 2011-519281, the lens is used to focus light, thus precluding the resolution from exceeding the diffraction limit of light. Thus, the structure of an optical absorber that is smaller than the wavelength of light cannot be imaged using the photoacoustic technique. Obtaining a resolution exceeding the diffraction limit of light needs the focusing of the light to a degree equal to or higher than the diffraction limit. It is generally known that near-field light can be focused to a degree equal to or higher than the diffraction limit.

Japanese Patent Application Laid-open No. 2007-307007 uses near-field light to obtain only a photoacoustic signal from an object located in the neighborhood of a light source. However, a configuration according to Japanese Patent Application Laid-open No. 2007-307007 fails to focus near-filed light and thus to achieve imaging at an increased resolution.

An example of an imaging technique using near-field light is scanning near field optical microscopy (SNOM). SNOM involves placing a probe generating near-field light closer to a sample, irradiating only a very small area equal to or smaller than, in size, the wavelength of the near-field light, and detecting scattering light and fluorescence from the sample. This operation is performed during scanning to obtain a two-dimensional image of the sample at a resolution equal to or smaller than the wavelength. However, SNOM involves significant scattering particularly in the case of the living tissue and the like and thus fails to directly measure the absorption of near-field light for a sample with a low transmittance. As a result, SNOM fails to obtain the distribution of absorption spectrum of the sample and functional information on the sample calculated from optical absorption information.

With the foregoing in view, it is an object of the present invention to provide a technique for enabling photoacoustic imaging at a resolution equal to or smaller than the wavelength of light.

The present invention provides an object information acquiring apparatus comprising:

a first light source configured to irradiate an object with irradiation light;

an output unit to which the irradiation light is guided and which comprises an opening smaller than a wavelength of the irradiation light;

a probe configured to detect an acoustic wave generated when the object absorbs near-field light output by the output unit; and

a signal processing unit configured to acquire information on an interior of the object from the acoustic wave detected by the probe.

An aspect of the present invention can provide a technique for enabling photoacoustic imaging at a resolution equal to or smaller than the wavelength of light.

Further features of the present invention will become apparent from the following description of exemplary embodiments with reference to the attached drawings.

### BRIEF DESCRIPTION OF THE DRAWINGS

FIG. 1 is a diagram showing an example of a configuration of a biological information imaging apparatus according to an embodiment of the present invention;

FIG. 2 is a diagram showing another example of a configuration of a biological information imaging apparatus according to an embodiment of the present invention;

FIG. 3 is a diagram showing an example of a configuration of a probe according to an embodiment of the present invention; and

FIG. 4 is a diagram showing another example of a configuration of a probe according to an embodiment of the present invention.

### DESCRIPTION OF THE EMBODIMENTS

Preferred embodiments of the present invention will be described below with reference to the drawings. However, the dimensions, materials, shapes, relative arrangements, and the like of components described below should be varied as appropriate according to the configuration of an apparatus to which the present invention is applied as well as various conditions, and are not intended to limit the scope of the present invention to the description below.

A object information acquiring apparatus according to embodiments of the present invention includes an apparatus utilizing a photoacoustic effect and which irradiates an object with light (electromagnetic wave) to receive an acoustic wave generated in the object, thus acquiring image data corresponding to object information.

For such an object information acquiring apparatus, the object information acquired refers to the distribution of sources of acoustic waves resulting from optical irradiation, the distribution of initial sound pressure in the object, or an optical energy absorption density distribution, an absorption coefficient distribution, or the distribution of the concentration of a substance forming a tissue; the optical energy absorption density distribution, the absorption coefficient distribution, and the substance concentration distribution are derived from the distribution of the initial sound pressure. The substance concentration distribution refers to, for example, an oxygen saturation distribution or an oxygenated and reduced hemoglobin concentration distribution.

The acoustic wave as used herein is typically an ultrasound wave and includes a sound wave, an ultrasound wave, and an elastic wave referred to as an acoustic wave. An acoustic wave generated by the photoacoustic effect is referred to as a photoacoustic wave or an optical ultrasound wave.

A biological information imaging apparatus in which the object information acquiring apparatus according to embodiments of the present invention is applied to the living tissue will be described by way of example. However, a measurement target according to embodiments of the present invention is not limited to the living tissue.

### Embodiment 1

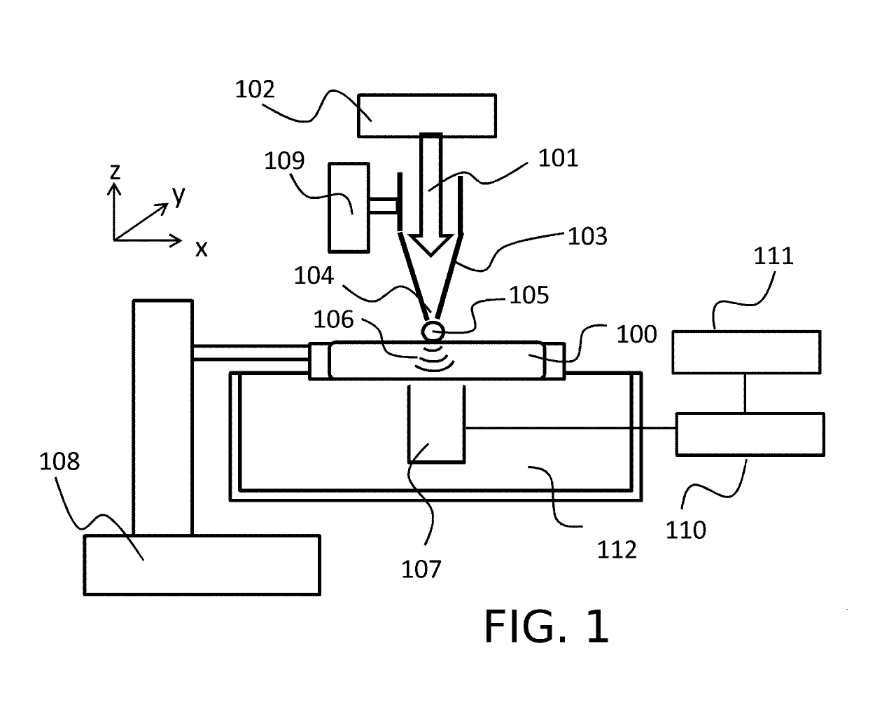

FIG. 1 shows a diagram illustrating an example of configuration of a biological information imaging apparatus according to the present embodiment. The biological information imaging apparatus according to the present embodiment enables imaging of the distribution of an optical characteristic value for the living tissue and the distribution of the concentration of a substance forming the living tissue which distribution is obtained from information in the distribution of the optical characteristic value.

A photoacoustic imaging apparatus according to the present embodiment includes a light source 102 (excitation light source) that outputs irradiation light 101 delivered to an object 100. The irradiation light 101 is guided to the object 100 through an optical probe 103 and becomes near-field light 105 at an opening 104 (a probe opening according to the present embodiment). The photoacoustic imaging apparatus according to the present embodiment further includes a probe 107 which detects a photoacoustic wave 106 generated by an optical absorber in the object 100 by absorbing a part of the energy of the near-field light 105 and which converts the photoacoustic wave 106 into an electric signal. The photoacoustic imaging apparatus according to the present embodiment further includes a scanning unit 108 that scans the object 100 and a movement mechanism 109 that moves the optical probe 103 in a Z direction. The near-field light is output through the opening, and thus, the opening corresponds to an output unit according to embodiments of the present invention.

The photoacoustic imaging apparatus according to the present embodiment further includes a signal processing unit 110 that analyzes an electric signal and generates image data such as information on the distribution of an optical characteristic value which is original data allowing corresponding images to be displayed to a user. Furthermore, an image display unit 111 is a device that displays the results of processing by the signal processing unit.

The optical probe 103 may guide the irradiation light 101 to the object 100 and may be an optical fiber. In this case, the opening 104 has a diameter smaller than the wavelength of the irradiation light 101. When an optical fiber is used as the optical probe 103, the opening 104 may be formed by sharpening the optical fiber by chemical etching and then coating the neighborhood of the opening with metal to produce a very small opening.

Although the diameter of the opening 104 has been referred to, the opening 104 may have a shape other than a circle. For example, when the opening 104 is square or rectangular, the opening 104 is set to be smaller, on a side, than the wavelength of the irradiation light 101.

An example of configuration of the photoacoustic imaging apparatus according to the present embodiment will be described below in detail.

The irradiation light 101 used has a wavelength at which the light is absorbed by a particular one of the components forming the object 100. The irradiation light 101 may be pulsed light. Preferred pulsed light has a duration of several nanoseconds and a wavelength of 400 nm or more and 1,600 nm or less.

The light source 102 generating the irradiation light 101 is preferably a laser but may be a light emitting diode instead of the laser. The laser may be any of various lasers such as a solid laser, a gas laser, a dye laser, and a semiconductor laser. A difference in the distribution of an optical characteristic value caused by the wavelength can be measured using a dye laser or an optical parametric oscillator (OPO) that enables conversion of an oscillating wavelength, or a titanium-sapphire laser.

The optical probe 103 is moved closer to the object 100 by the movement mechanism 109. Alignment needs to be carried out so as to place a surface of the object 100 in the area of the near-field light 105. Thus, the movement mechanism 109 is preferably able to finely move the optical probe 103. For example, an actuator based on a piezoelectric element may be used.

The probe 107 detects the photoacoustic wave 106 generated by the optical absorber in the object 100 by absorbing a part of the energy of the near-field light 105 and converts the photoacoustic wave 106 into an electric signal.

The probe may be any sound wave detector provided that the probe can detect a photoacoustic signal and may be, for example, a transducer using a piezoelectric phenomenon, a transducer using optical resonance, or a transducer using a change in capacity.

The probe 107 preferably uses a large bandwidth in order to receive frequency components of the photoacoustic wave 106. The probe preferably has a center frequency of 50 MHz or higher in order to receive the photoacoustic wave 106 from a very small optical absorber.

The probe 107 is preferably a focus probe designed to focus sound. The focus sound may be a probe including an acoustic lens for focusing sound. However, any probe that can focus sound may be used, and for example, a probe with a recessed reception surface may be used.

An acoustic coupling medium is desirably provided between the probe 107 and the object 100 to suppress reflection of an acoustic wave. In FIG. 1, the probe 107 is disposed in a water bath with water 112 filled therein, and a bottom surface of the object is immersed in water 112 to suppress reflection of an acoustic wave. Alternatively, an impedance matching gel may be provided between the probe 107 and the object 100.

When an electric signal obtained from the probe 107 is small, an amplifier is preferably used to amplify the intensity of the signal.

The probe 107 detects the photoacoustic wave 106 generated by the object 100 to convert the photoacoustic wave 106 into an electric signal while scanning the object 100 in an X direction and a Y direction. Thus, the two-dimensional distribution of the photoacoustic wave 106 generated by the object 100 is obtained. However, the same effect can be exerted by allowing the photoacoustic wave 106 to be detected at a plurality of positions, and thus, the probe 107 and the optical probe 103 may be synchronously scanned in the same direction along the surface of the object 100.

Furthermore, the two-dimensional distribution can be obtained without any scan by using an arrayed probe as the probe 107.

The signal processing unit 110 according to the present embodiment calculates the position and size of the absorber in the object or the distribution of an optical characteristic value such as the distribution of the optical absorption coefficient or the distribution of the amount of optical energy accumulated, based on the two-dimensional distribution of electric signals obtained by the probe 107.

Possible image processing for obtaining the distribution of an optical characteristic value from the electric signals obtained at the plurality of positions involves carrying out envelope detection using a Hilbert transform and then converting a time axis for a sound arrival time into a space axis in a sound focus direction to obtain three-dimensional position data.

The signal processing unit 110 may be any signal processing unit provided that the signal processing unit 110 can store the intensity of the acoustic wave and temporal changes in intensity and convert the intensity and the changes in intensity into data for the distribution of an optical characteristic value using arithmetic means. For example, a data acquisition (DAQ) system and a computer that can analyze data stored by the DAQ system may be used as the signal processing unit 110.

When light with a plurality of wavelengths is used, optical coefficients for the interior of the object are calculated, and the resultant values are compared with wavelength dependence inherent in substances (glucose, collagen, oxygenated and reduced hemoglobin, and the like) forming the living tissue. This also enables imaging of the distribution of the concentration of a substance forming the living organism.

Furthermore, the embodiment of the present invention includes the image display unit 111 that displays image information obtained by signal processing.

In what is called a transmissive configuration based on SNOM in which the near-field light and the detector are disposed at the opposite positions with respect to the sample as shown in FIG. 1, the living tissue or the like causes light to scatter significantly, making a less transmissive sample difficult to image. On the other hand, according to the present embodiment, the detected medium is an ultrasound wave and is thus transmitted through the living tissue. Hence, information on the interior of the object can be acquired even though the opening and the detector are disposed at the opposite positions.

Moreover, in accordance with SNOM, light is incident and is detected, and thus, extracting only information on an area to be viewed is difficult unless any measure is taken, for example, the wavelength is converted using a phosphor or the like. On the other hand, according to the present embodiment, converting light into a photoacoustic wave enables only the information from the optical absorber to be obtained. This enables imaging of an optical absorption distribution that is otherwise difficult to obtain in accordance with SNOM.

The use of such an imaging apparatus as illustrated in the embodiment allows the resolution of photoacoustic imaging to be increased to a value equal to or larger than an optical diffraction limit. Thus, the optical absorption distribution and the structure of the optical absorber can be imaged at a resolution equal to or smaller than the wavelength of light.

### Embodiment 2

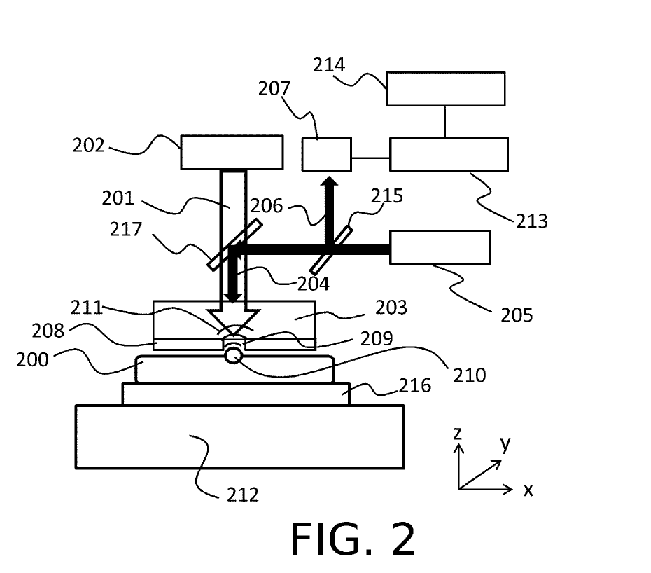

FIG. 2 shows a diagram illustrating an example of configuration of a biological information imaging apparatus according to the present embodiment. The biological information imaging apparatus according to the present embodiment enables imaging of the distribution of an optical characteristic value for the living tissue and the distribution of the concentration of a substance forming the living tissue obtained from the information in the distribution of the optical characteristic value.

The photoacoustic imaging apparatus according to the present embodiment includes a first light source 202 (excitation light source) that outputs irradiation light 201 delivered to an object 200. The photoacoustic imaging apparatus according to the present embodiment further includes a Fabry-Perot sensor (Fabry-Perot resonator) 203 and a second light source 205 (measurement light source) that outputs measurement light 204 incident on the Fabry-Perot sensor 203. The measurement light 204 has a wavelength different from the wavelength of the irradiation light 201. Reflected light 206 corresponding to the measurement light 204 reflected by the Fabry-Perot sensor 203 is guided to a photodiode 207.

A light-blocking layer 208 that blocks the irradiation light 201 is provided on a bottom surface of the Fabry-Perot sensor 203. Furthermore, an opening 209 formed on the light-blocking layer 208 is provided with no light-blocking member. The size of the opening 209 is smaller than the wavelength of the irradiation light 201. The irradiation light 201 becomes near-field light 210 at the opening 209.

When a photoacoustic wave 211 is generated by an optical absorber in the object 200 by absorbing a part of the energy of the near-field light 210 and enters the Fabry-Perot sensor 203, the amount of light in the reflected light 206 changes. The photodiode 207 converts the amount of light in the reflected light 206 into an electric signal.

The photoacoustic imaging apparatus according to the present embodiment further includes a scanning unit 212 that scans the object 200 in the X direction and the Y direction.

The photoacoustic imaging apparatus according to the present embodiment further includes a signal processing unit 213 that analyzes an electric signal and generates image data such as information on the distribution of an optical characteristic value which is original data allowing corresponding images to be displayed to a user. Furthermore, an image display unit 214 is a device that displays the results of processing by the signal processing unit.

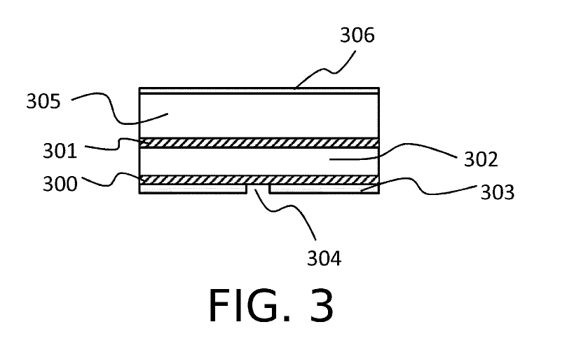

FIG. 3 is a diagram illustrating the cross-sectional structure of the Fabry-Perot sensor 203 according to the present embodiment.

A first mirror 300 and a second mirror 301 reflect the measurement light 204 in order to function as a Fabry-Perot sensor. Preferably, the first mirror 300 and the second mirror 301 have a reflectance of 90% or more with respect to the wavelength region of the measurement light 204 in order to increase the sensitivity of a probe. On the other hand, the first mirror 300 and the second mirror 301 transmit the irradiation light 201 so that the irradiation light 201 is transmitted through the Fabry-Perot sensor and becomes the near-field light 210 at the opening 209. Preferably, to efficiently irradiate the object 200 with the near-field light 210, the first mirror 300 and the second mirror 301 have a transmittance of 90% or more with respect to the wavelength region of the irradiation light 201.

A dielectric multilayer film may be used as a material for the first mirror 300 and second mirror 301 described above.

A light-blocking layer 303 is provided on the first mirror 300. The light-blocking layer 303 blocks the irradiation light 201. As the light-blocking member 303, a reflecting layer which reflects the irradiation light 201 or an absorbing layer which absorbs the irradiation light 201 can be utilized. Preferably, the light-blocking layer 303 has a reflectance of 90% or more with respect to the wavelength region of the irradiation light 201. For example, a gold film or an aluminum film may be used as the light-blocking layer 303 functioning as the reflection layer described above. The gold film may be formed by being deposited on the first mirror 300. Moreover, a member which has not less than 90% of absorption factor for the wavelength area of the irradiation light can be utilized as the light-blocking layer 303. As such light-blocking layer functioning as the absorbing layer, for example, carbon film can be used. Here, as the light-blocking layer 303, the reflecting layer is preferable to the absorbing layer in order to suppress acoustic waves generating in the light-blocking layer.

Furthermore, an opening 304 formed on the light-blocking layer 303 is provided with no light-blocking member. The opening 304 has a diameter smaller than the wavelength of the irradiation light 201. For example, photolithography can be used as a technique for producing the opening 304.

By the way, if no members are arranged at the opening 304, the reception characteristics for the acoustic wave at an area of the first mirror 300, the area contacts the opening 304, may be different from the reception characteristics at other areas of the first mirror 300. Therefore compensation of the membrane characteristics is expected so as to reduce the gap of the reception characteristics of each areas of the first mirror 300.

Concretely, a compensation member can be arranged at the opening 304. Here, an acoustic impedance of the compensation member is preferably similar to an acoustic impedance of the light-blocking member 303. Meanwhile, the acoustic impedance of the compensation member is preferably within ±10% from the acoustic impedance of the light-blocking member 304. At the same time, the compensation member is preferably made of materials which is transparent to the irradiation light 201. Moreover, preferable transmittance of the compensation member to the wavelength area of the irradiation light 201 is not less than 90%. For example, glass can be used as the compensation member.

Although the diameter of the opening 304 has been referred to, the opening 304 may have a shape other than a circle. For example, when the opening 304 is square or rectangular, the opening 304 is set to be smaller, on a side, than the wavelength of the irradiation light 201.

A spacer film 302 is present between the first mirror 300 and the second mirror 301. The spacer film 302 is preferably significantly strained when the photoacoustic wave 211 enters the Fabry-Perot sensor 203. The spacer film 302 may be, for example, an organic polymer film. The organic polymer film may be, for example, parylene, SU8, or polyethylene. Any film may be used provided that the film is deformed upon receiving a sound wave, and thus, the spacer film 302 may be an inorganic film.

A substrate 305 on which the second mirror 301 is deposited may be glass or acrylic. In this case, the substrate 305 is preferably shaped in a wedge in order to reduce the adverse effect of optical interference in the substrate 305.

Moreover, the substrate 305 preferably has an AR coating 306 in order to avoid reflection of light from a surface of the substrate 305. The AR coating preferably has a reflectance of 2% or less for the irradiation light 201 and the measurement light 204.

An example of configuration of the biological information imaging apparatus according to the present embodiment will be described below in detail.

Irradiation light and an image display unit according to the present embodiment may be similar to the irradiation light and image display unit according to Embodiment 1 and will thus not be described below in detail.

The Fabry-Perot sensor 203 and the object 200 are moved closer to each other by the movement mechanism 216. Alignment needs to be carried out in the Z direction so as to place a surface of the object 200 in the area of the near-field light 210. Thus, the movement mechanism 216 is preferably able to finely move the object 200. For example, an actuator based on a piezoelectric element may be used. Furthermore, although the movement mechanism 216 moves the object 200 according to the present embodiment, the Fabry-Perot sensor 203 may be moved for alignment in the Z direction.

The photodiode 207 detects a change in the amount of light in the reflected light 206 from the Fabry-Perot sensor 203 which change is caused by the photoacoustic wave 211 generated by the optical absorber in the object 200 by absorbing a part of the energy of the near-field light 210. The photodiode 207 then converts the change into an electric signal.

For the Fabry-Perot sensor 203, the optimal wavelength of the measurement light 204, which allows the sensitivity of the sensor 203 to be maximized, varies depending on temperature and the thickness of the spacer layer. The second light source 205 outputting the measurement light 204 can preferably change the wavelength of the measurement light 204 so as to obtain the optimal wavelength.

Furthermore, for improved sensitivity, the measurement light 204 preferably has a small spectral line width. The light source 205 as described above may be a DBR laser, a DFB laser, a VCSEL laser, an external cavity laser, or the like.

Furthermore, a mirror 217 and a half mirror 215 are used to guide the measurement light 204 and the reflection light 206 to the object 200 and the photodiode 207, respectively. A wavelength plate and a polarizing mirror may be used instead of the half mirror 215. Furthermore, instead of these optical components, a fiber coupler or the like may be used.

An acoustic coupling medium is desirably provided between the object 200 and the Fabry-Perot sensor 203 to suppress reflection of an acoustic wave. For example, an impedance matching gel may be used.

When an electric signal obtained from the photodiode 207 is small, an amplifier is preferably used to amplify the intensity of the signal.

The photodiode 207 detects a change in the amount of light in the reflected light 206 which is caused by the photoacoustic wave 211 generated by the object 200 to convert the change into an electric signal while scanning the object 200 in the X direction and the Y direction by the scanning unit 212. Thus, the two-dimensional distribution of the photoacoustic wave 211 generated by the object 100 is obtained.

However, the same effect can be exerted by allowing the photoacoustic wave 211 to be detected at a plurality of positions, and thus, with the object 200 fixed, the Fabry-Perot sensor 203, the irradiation light 201, and the measurement light 204 may be synchronously scanned in the same direction along the surface of the object 200. In this case, a galvanometer scanner may be used as means for scanning the irradiation light 201 and the measurement light 204.

Alternatively, a plurality of the openings 209 may be formed on the light-blocking layer 208, and a galvanometer scanner or the like may be used to scan the irradiation light 201 and the measurement light 204. This method allows the two-dimensional distribution of the photoacoustic wave 211 generated by the object 200 without scanning the Fabry-Perot sensor 203 or the object 200.

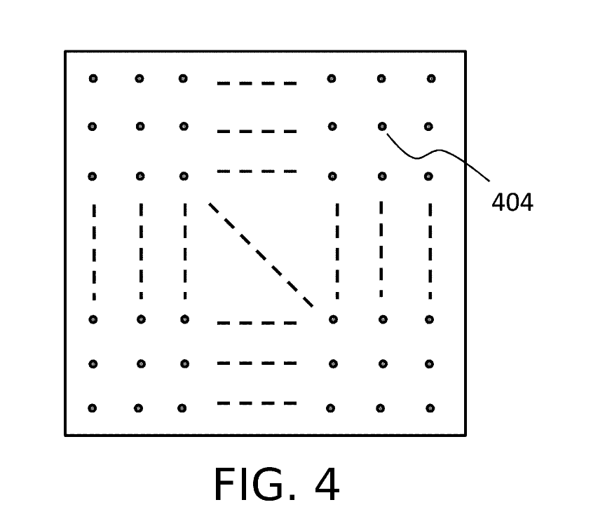

Such an example as described above is shown in FIG. 4. FIG. 4 is a diagram showing the Fabry-Perot sensor 203 as seen from the light-blocking layer 208 side. Openings 404 are two-dimensionally formed at regular intervals. Dotted lines indicate that a large number of openings are preset at regular intervals. In this case, the two-dimensional distribution of the photoacoustic wave 211 generated by the object 200 can be obtained by scanning the irradiation light 201 and the measurement light 204 and detecting a change in the amount of light in the reflected light 206 for each of the openings 404.

The two-dimensional distribution of the photoacoustic wave 211 may also be obtained by widening the irradiation light 201 so that the irradiation light 201 covers the range of the two-dimensionally arranged openings 404 and scanning the measurement light 204.

Moreover, a change in the amount of light in the reflected light 206 is preferably detected by widening the irradiation light 201 and the measurement light 204 so that the irradiation light 201 and the measurement light 204 cover the range of the two-dimensionally arranged openings 404 and using a two-dimensional optical sensor such as a CCD instead of the photodiode 207. This enables the two-dimensional distribution of the photoacoustic wave 211 to be obtained without scanning the irradiation light 201 and the measurement light 204.

The signal processing unit 213 according to the present embodiment calculates the position and size of the absorber in the object or the distribution of an optical characteristic value such as the distribution of the optical absorption coefficient or the distribution of the amount of optical energy accumulated, based on the two-dimensional distribution of electric signals obtained by the photodiode 207.

Possible image processing for obtaining the distribution of an optical characteristic value from the electric signals obtained at the plurality of positions involves carrying out envelope detection using a Hilbert transform and then converting a time axis for a sound arrival time into a space axis in a sound focus direction to obtain three-dimensional position data.

Any signal processing unit 213 may be used provided that the signal processing unit can store the intensity of the photoacoustic wave and temporal changes in the intensity and convert the intensity and the changes in intensity into data for the distribution of an optical characteristic value using arithmetic means. For example, a data acquisition (DAQ) system and a computer that can analyze data stored by the DAQ system may be used as the signal processing unit 213.

When light with a plurality of wavelengths is used as the irradiation light 201, optical coefficients for the interior of the object are calculated, and the resultant values are compared with wavelength dependence inherent in substances (glucose, collagen, oxygenated and reduced hemoglobin, and the like) forming the living tissue. This also enables imaging of the distribution of the concentration of a substance forming the living organism.

Furthermore, the embodiment of the present invention includes the image display unit 214 that displays image information obtained by the signal processing.

The present embodiment, using the Fabry-Perot sensor, has several advantages compared to Embodiment 1.

A first advantage is that, according to the present embodiment, the optical characteristics of the mirror and the light-blocking layer are improved to provide what is called a reflective configuration in which the probe and the near field are arranged on the same side with respect to the object. When a transmissive configuration is used as is the case with Embodiment 1, a thick object increases the distance between the probe and the source of a photoacoustic wave. Then, a photoacoustic wave generated in a near-field area of the object surface is attenuated during propagation of a sound wave due to the spread and absorption of the sound and cannot be detected. On the other hand, the reflective configuration can detect a photoacoustic wave generated in the near-field area of the object surface regardless of the thickness of the object.

Furthermore, the near field can be formed immediately below the probe. This allows a photoacoustic wave generated immediately below the probe to be detected, reducing the distance between the source of a photoacoustic wave and a reception surface of the probe. Thus, the attenuation of the photoacoustic wave can be minimized to improve SN.

A second advantage is that the probe and the opening, which allows the near field to be generated, can be formed into a single device. The device can be produced by a process, allowing the near-field photoacoustic probe to be miniaturized.

A third advantage is that the Fabry-Perot sensor uses a large bandwidth. This enables a photoacoustic wave from a very small optical absorber to be received. The use of the imaging apparatus illustrated in the embodiments increases the resolution of photoacoustic imaging by making the resolution equal to or higher than the diffraction limit of light. Therefore, the optical absorption distribution and the structure of the optical absorber can be imaged at a resolution equal to or smaller than the wavelength of light.

The examples of configuration of the biological information imaging apparatus for which the object is a living organism have mainly been described herein. This enables imaging of the distribution of an in vivo optical characteristic value and the distribution of the concentration of a substance forming the living tissue which distribution is obtained from information in the distribution of the in vivo optical characteristic value, in order to allow diagnosis of tumors, vascular vessel diseases, and the like, and chemical treatment follow-ups. Thus, the biological information imaging apparatus can be utilized as a medical image diagnosis apparatus.

Moreover, those skilled in the art can easily apply the biological information imaging apparatus to non-destructive inspection in which the object is a non-biological substance.

As described above, the embodiments of the present invention can be widely used as inspection apparatuses.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-193844, filed on Sep. 4, 2012, which is hereby incorporated by reference herein in its entirety.

What is claimed is:

1. An object information acquiring apparatus comprising:
a first light source configured to irradiate an object with irradiation light;
an output unit to which the irradiation light is guided and which comprises an opening smaller than a wavelength of the irradiation light;
a probe configured to detect an acoustic wave generated when the object absorbs near-field light output by the output unit; and
a signal processing unit configured to acquire information on an interior of the object from the acoustic wave detected by the probe.

2. The object information acquiring apparatus according to claim 1, wherein the probe is a probe using a Fabry-Perot sensor,
the object information acquiring apparatus further comprises a second light source configured to output measurement light for detecting a change in reflectance of the Fabry-Perot sensor,
the probe comprises a first mirror and a second mirror transmitting the irradiation light and reflecting the measurement light and a light-blocking layer provided on the first mirror and blocking the irradiation light, and
the output unit is provided on the light-blocking layer.

3. The object information acquiring apparatus according to claim 2, wherein the probe comprises a plurality of the output units.

4. The object information acquiring apparatus according to claim 2, wherein the light-blocking layer has a reflectance of 90% or more with respect to a wavelength region of the irradiation light.

5. The object information acquiring apparatus according to claim 2, wherein the first mirror and the second mirror have a transmittance of 90% or more with respect to a wavelength region of the irradiation light.

6. The object information acquiring apparatus according to claim 2, wherein the first mirror and the second mirror have a reflectance of 90% or more with respect to a wavelength region of the measurement light.

7. The object information acquiring apparatus according to claim 2, wherein the output unit is formed by photolithography.

8. The object information acquiring apparatus according to claim 1, further comprising an optical fiber configured to guide the irradiation light,
wherein the output unit is formed by sharpening the optical fiber.

9. The object information acquiring apparatus according to claim 8, wherein the probe is a focus probe.

10. The object information acquiring apparatus according to claim 9, wherein the probe comprises an acoustic lens for focusing sound.

11. The object information acquiring apparatus according to claim 8, wherein the probe is disposed opposite the output unit with respect to the object.

12. The object information acquiring apparatus according to claim 1, further comprising a scanning unit configured to move the object.

13. The object information acquiring apparatus according to claim 1, wherein the output unit scans the object and irradiates the object with near-field light at a plurality of positions, and
the probe scans the object in synchronism with the output unit to detect an acoustic wave.

14. The object information acquiring apparatus according to claim 1, wherein the output unit is shaped in a circle with a diameter smaller than the wavelength of the irradiation light.

15. The object information acquiring apparatus according to claim 1, further comprising an image display unit configured to display image data generated based on the information on the interior of the object acquired by the signal processing unit.
